(19) United States
(12) Patent Application Publication
(10) Pub. No.: US 2026/0192080 A1
(43) Pub. Date: Jul. 9, 2026

(54) INTEGRATION OF WEARABLE HEALTH TRACKING DATA WITH SLEEP THERAPY DEVICES AND RELATED METHODS

(71) Applicant: RESMED PTY LTD, Bella Vista (AU)

(72) Inventors: Liam HOLLEY, Sydney (AU); Ryan Michael KIRKPATRICK, Sydney (AU); Stephanie KLINKENBERG, Sydney (AU); Andrew William GILLETT, Sydney (AU)

(21) Appl. No.: 19/374,896

(22) Filed: Oct. 30, 2025

(30) Foreign Application Priority Data
Nov. 1, 2024 (AU) 2024903573

(51) Int. Cl. A61M 21/02; A61M 16/00; G16H 40/67

(52) U.S. Cl. CPC A61M 21/02; A61M 16/024; G16H 40/67; A61M 2230/10; A61M 2230/205

(57) ABSTRACT

A method for providing personalized recommendations for improving sleep health using data from wearable health tracking devices and sleep therapy treatment devices comprises obtaining, by a system, user health data from one or more wearable health tracking devices, obtaining, by the system, user sleep therapy data from one or more sleep therapy treatment devices, combining, by the system, the user health data and the user sleep therapy data to generate combined user data, and providing, by the system, personalized recommendations for improving sleep health based on the combined user data, wherein the personalized recommendations include at least one of sleep condition screening, at least one sleep therapy treatment, at least one additional sleep therapy treatment, or at least one sleep hygiene action.

### CROSS-REFERENCE TO RELATED APPLICATIONS

This application claims priority to Australian Patent Provisional Application No. 2024903573 filed November 1, 2024, the entire contents of which are hereby incorporated by reference.

### TECHNICAL FIELD

The invention relates to the integration of wearable health tracking data with sleep therapy devices to enhance sleep condition screening, treatment recommendations, and therapy parameter adjustments.

### BACKGROUND

Wearable health tracking devices have become increasingly popular in recent years, offering users the ability to monitor various aspects of their health and wellness, such as physical activity, heart rate, and sleep patterns. These devices, including smartwatches, smart rings, and smart bands, provide valuable insights into a user's daily habits and overall health. Concurrently, sleep disorders have become a significant public health concern, affecting millions of individuals worldwide. Conditions such as sleep apnea, insomnia, and restless leg syndrome can severely impact a person's quality of life, leading to increased risks of chronic health issues like cardiovascular disease, obesity, and diabetes.

Sleep therapy treatment devices, such as Continuous Positive Airway Pressure (CPAP) machines, are commonly used to manage sleep disorders, particularly sleep apnea. These devices help maintain open airways during sleep, improving sleep quality and reducing associated health risks. However, the effectiveness of sleep therapy can vary significantly among individuals, necessitating personalized treatment plans. The integration of data from wearable health tracking devices with sleep therapy treatment devices presents an opportunity to enhance the personalization of sleep therapy, offering tailored recommendations and adjustments to treatment parameters based on comprehensive data analysis. This approach addresses the growing need for more effective and individualized sleep disorder management solutions.

### SUMMARY

In accordance with embodiments, a method is provided for personalized recommendations for improving sleep health using data from wearable health tracking devices and sleep therapy treatment devices. The method involves obtaining user health data from wearable health tracking devices and user sleep therapy data from sleep therapy treatment devices. The system combines this data to generate combined user data and provides personalized recommendations for improving sleep health. These recommendations may include sleep condition screening, sleep therapy treatments, additional sleep therapy treatments, or sleep hygiene actions.

In accordance with other embodiments, a method is provided for generating modifications to sleep therapy treatment parameters using data from wearable health tracking devices and sleep therapy treatment devices. The method includes obtaining user health data and user sleep therapy data, combining this data to generate combined user data, and generating modifications to sleep therapy treatment parameters based on this data. The modifications are then applied to a sleep therapy device.

In yet other embodiments, the method further includes evaluating the effectiveness of sleep therapy treatment or the quality of the user's sleep using the user health data obtained from wearable health tracking devices.

In accordance with additional embodiments, the wearable health tracking devices may include a smart watch, a smart ring, or a smart band.

In other embodiments, the sleep therapy treatment devices may include a continuous positive airway pressure (CPAP) device.

In further embodiments, the method includes comparing user health data with health data from other users of sleep therapy treatment devices to make personalized recommendations.

In yet another embodiment, the method includes referring the user to a marketplace of sleep therapy providers or sleep therapy device providers based on the personalized recommendations.

In additional embodiments, the method includes suggesting a specific provider based on the personalized recommendations.

In other embodiments, the method includes ranking providers based on the personalized recommendations.

In accordance with further embodiments, the method includes suggesting or making changes to sleep therapy treatment parameters based on user health data obtained from wearable health tracking devices while the user is using a sleep therapy treatment device.

In yet other embodiments, the method includes suggesting or making changes to sleep therapy treatments based on user health data obtained from wearable health tracking devices while the user is not using a sleep therapy treatment device.

In additional embodiments, the method includes suggesting an additional sleep therapy treatment based on user health data obtained from wearable health tracking devices while the user is using a sleep therapy treatment device.

In other embodiments, the method includes monitoring the effectiveness of sleep therapy when the user is using a sleep therapy device without electronic monitoring capabilities by using user health data obtained from wearable health tracking devices.

In accordance with embodiments, a system is provided for personalized recommendations for improving sleep health using data from wearable health tracking devices and sleep therapy treatment devices. The system comprises processors and memory storing instructions to obtain user health data and user sleep therapy data, combine this data to generate combined user data, and provide personalized recommendations for improving sleep health.

In accordance with other embodiments, a system is provided for generating modifications to sleep therapy treatment parameters using data from wearable health tracking devices and sleep therapy treatment devices. The system includes processors and memory storing instructions to obtain user health data and user sleep therapy data, combine this data to generate combined user data, generate modifications to sleep therapy treatment parameters, and apply these modifications to a sleep therapy device.

In yet other embodiments, the system further evaluates the effectiveness of sleep therapy treatment or the quality of the user's sleep using user health data obtained from wearable health tracking devices.

In accordance with additional embodiments, the wearable health tracking devices may include a smart watch, a smart ring, or a smart band.

In other embodiments, the sleep therapy treatment devices may include a continuous positive airway pressure (CPAP) device.

In further embodiments, the system compares user health data with health data from other users of sleep therapy treatment devices to make personalized recommendations.

In yet another embodiment, the system refers the user to a marketplace of sleep therapy providers or sleep therapy device providers based on the personalized recommendations.

In accordance with further embodiments, the system suggests or makes changes to sleep therapy treatment parameters based on user health data obtained from wearable health tracking devices while the user is using a sleep therapy treatment device.

In yet other embodiments, the system suggests or makes changes to sleep therapy treatments based on user health data obtained from wearable health tracking devices while the user is not using a sleep therapy treatment device.

In additional embodiments, the system suggests an additional sleep therapy treatment based on user health data obtained from wearable health tracking devices while the user is using a sleep therapy treatment device.

In other embodiments, the system monitors the effectiveness of sleep therapy when the user is using a sleep therapy device without electronic monitoring capabilities by using user health data obtained from wearable health tracking devices.

### BRIEF DESCRIPTION OF THE DRAWINGS

FIG. 1 illustrates, in a flowchart, operations for providing personalized sleep health recommendations based on combined user data.

FIG. 2 illustrates, in a flowchart, operations for modifying sleep therapy parameters using combined user data.

FIG. 3 illustrates a system integrating devices for sleep health recommendations.

FIG. 4 illustrates a system for modifying and evaluating sleep health recommendations.

FIG. 5 is an overview of pipeline utilizing ECG signals for the detection of sleep apnea.

FIG. 6 is a sequential block diagram of signal preprocessing.

FIG. 7 is an architecture of a BAFNet.

### DETAILED DESCRIPTION

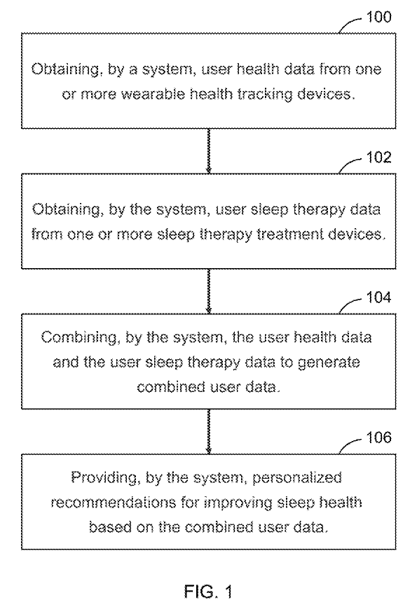

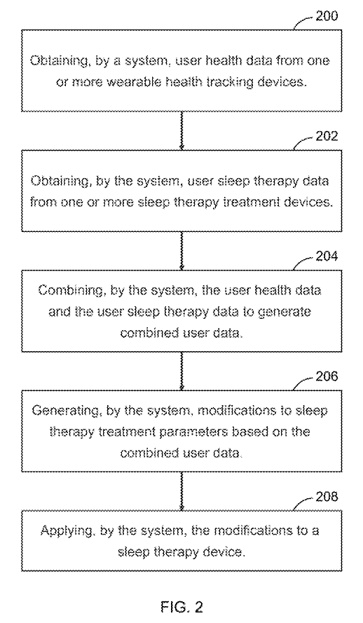

FIG. 1 is a flowchart illustrating a method for obtaining user health data from wearable health tracking devices. The system may obtain user health data from devices such as smart watches, smart rings, or smart bands, which collect health metrics like heart rate, activity levels, and sleep patterns. This data serves as input for further analysis and processing. The system may use this data to assess the user's health status and identify areas for improvement in sleep health. Integration of data from these devices may enable personalized recommendations for sleep condition screening, possible sleep therapy treatments, or sleep hygiene actions. The system may also compare the user's health data with data from other users to enhance the accuracy and relevance of the recommendations. This comparison may help identify users with similar characteristics, allowing for more tailored recommendations. Additionally, the system may refer the user to a marketplace of sleep therapy providers or device providers based on the recommendations, assisting the user in selecting the most suitable option. The process may contribute to improving the user's sleep health by leveraging wearable health tracking devices and the system's analytical capabilities.

The system may obtain user sleep therapy data from devices such as CPAP machines, designed to assist with sleep-related issues. Sleep therapy device may be This data may include metrics like usage duration, pressure settings, and compliance information, which are used to assess therapy effectiveness. The system may integrate this data with other user health data to form a comprehensive view of the user's sleep health, enabling personalized recommendations or modifications to the sleep therapy regimen. The system's ability to process this data enables delivery of tailored sleep health solutions, potentially improving sleep quality and therapy outcomes.

The system may combine user health data and user sleep therapy data to generate combined user data. This involves integrating data from wearable health tracking devices with data from sleep therapy treatment devices. The combination of these data sets enables the creation of a comprehensive profile of the user's health and sleep patterns. This combined data serves as a foundation for generating personalized recommendations aimed at improving sleep health. The system may use this integrated data to provide recommendations for sleep condition screening, potential sleep therapy treatments, or sleep hygiene actions. The integration of diverse data sources enhances the accuracy and relevance of the recommendations, potentially leading to more effective sleep health interventions. The system may also leverage this combined data to compare the user's health data with that of other users, refining the recommendations based on similarities in health and sleep characteristics.

The system may provide personalized recommendations for improving sleep health based on the combined user data. These recommendations may include options such as sleep condition screening, potential sleep therapy treatments, or sleep hygiene actions. The system may compare the user's health data with data from other users to make these recommendations, helping identify users with similar characteristics for more tailored recommendations. Furthermore, the user may be referred to a marketplace of sleep therapy providers or device providers based on the recommendations. This referral may facilitate access to providers or devices that align with specific needs. Additionally, the system may suggest a specific provider based on the recommendations, assisting the user in selecting a provider that best suits their requirements. Providers may also be ranked based on the recommendations, offering a prioritized list of options. This ranking may guide the user towards the most suitable providers or devices. The system's ability to provide comprehensive and personalized recommendations may enhance the user's sleep health by offering targeted solutions and facilitating access to appropriate resources.

The process may obtain user health data from wearable health tracking devices. This data acquisition involves devices capable of monitoring various health metrics. The system may obtain user sleep therapy data from devices like CPAP machines. The integration of these data sources allows the system to combine the user health data and the user sleep therapy data to generate combined user data. In some examples, this combined data serves as a foundation for generating modifications to sleep therapy treatment parameters. In other examples, modifications may be suggested based solely on the health data. The system may then apply these modifications to a sleep therapy device, potentially enhancing treatment effectiveness. In some examples, the system may recommend the modifications to a health provider for approval prior to making the modification. Additionally, the system may evaluate the effectiveness of the sleep therapy treatment or the quality of the user's sleep using the user health data. This evaluation provides insights into the treatment's impact and informs further adjustments. Furthermore, the system may suggest or make changes to sleep therapy treatment parameters based on the user health data while the user is using a sleep therapy treatment device. In scenarios where the user is not using a sleep therapy treatment device, the system may still suggest or make changes to sleep therapy treatments based on the available health data from the wearable device. The system may also suggest an additional sleep therapy treatment based on the user health data while the user is using a sleep therapy treatment device. Lastly, the system may monitor the effectiveness of sleep therapy when the user is using a device without electronic monitoring capabilities by utilizing the user health data. This comprehensive approach ensures that the sleep therapy treatment is continuously optimized for the user's needs.

The system may obtain user sleep therapy data from devices such as CPAP machines. The data obtained may include parameters related to the user's sleep therapy, such as pressure settings, usage duration, and compliance data. The system may use this data to understand the user's current sleep therapy regimen and its effectiveness. This step is crucial for integrating the sleep therapy data with user health data obtained from wearable health tracking devices in subsequent steps. The integration of these data sets allows the system to generate a comprehensive view of the user's sleep health, which can be used to provide personalized recommendations or to modify sleep therapy treatment parameters. The system may also use this data to monitor the effectiveness of the sleep therapy, especially in cases where the sleep therapy device lacks electronic monitoring capabilities. This step serves as a foundation for further analysis and decision-making processes within the system, ultimately aiming to enhance the user's sleep health through tailored interventions.

The process may involve the combination of user health data and user sleep therapy data to generate combined user data. The system may use data from wearable health tracking devices alongside data from sleep therapy treatment devices. The integration of these data sources enables the system to form a holistic view of the user's health and sleep patterns. In some examples, the combined user data serves as a foundational element for subsequent steps, where modifications to sleep therapy treatment parameters may be generated. This integration facilitates the identification of patterns or anomalies in the user's sleep health, potentially leading to more informed and personalized recommendations. The system may employ algorithms to analyze the combined data, identifying correlations and trends that may not be apparent when considering the data sets in isolation. This process may be iterative, with the system continuously refining its analysis as new data becomes available. The ultimate goal of this step is to enhance the accuracy and relevance of the personalized recommendations provided to the user, thereby improving the overall effectiveness of the sleep therapy treatment.

The system may engage in generating modifications to sleep therapy treatment parameters based on the combined user data. This process involves the integration of user health data obtained from wearable health tracking devices and user sleep therapy data from sleep therapy treatment devices which may include current parameters. The combined user data serves as a foundation for identifying potential adjustments to the parameters of sleep therapy treatments. The system may analyze the combined data to discern patterns or anomalies that could suggest the need for modifications. These modifications aim at optimizing the effectiveness of the sleep therapy treatment, potentially enhancing the user's sleep quality or addressing specific sleep health issues. The system may employ algorithms or models to evaluate the combined data, considering various factors such as the user's health metrics, sleep patterns, and therapy device performance. The outcome of this analysis may lead to the generation of specific recommendations for modifying the treatment parameters, which may then be applied to the sleep therapy device to implement the suggested changes.

The process may involve the application of modifications to a sleep therapy device. This step is part of a broader system designed to enhance sleep health by utilizing data from wearable health tracking devices and sleep therapy treatment devices. The modifications applied to the sleep therapy device may be generated based on combined user data, which includes user health data and user sleep therapy data or based solely on the health data from the wearable health tracking device. The system may evaluate the effectiveness of sleep therapy treatment or the quality of the user's sleep using the user health data obtained from wearable health tracking devices. This evaluation informs the system's decision to suggest or make changes to sleep therapy treatment parameters. These changes may be based on the user health data obtained while the user is using a sleep therapy treatment device. Additionally, the system may conduct monitoring and/or suggest or make changes to sleep therapy treatments based on the user health data obtained while the user is not using a sleep therapy treatment device. For example, the user may only use comply with using a sleep therapy device for some parts of a night and may only be trackable with the sleep therapy device while using the device. The wearable device may supplement the data obtained by the sleep therapy device and enable a whole view of the user's sleep with and without the device.

Furthermore, the system may suggest an additional sleep therapy treatment based on the user health data obtained while the user is using a sleep therapy treatment device. The system may also monitor the effectiveness of sleep therapy when the user is using a device without electronic monitoring capabilities such as a mouthguard by utilizing the user health data. This comprehensive approach ensures that the sleep therapy treatment is tailored to the user's specific needs and may enhance the overall effectiveness of the treatment.

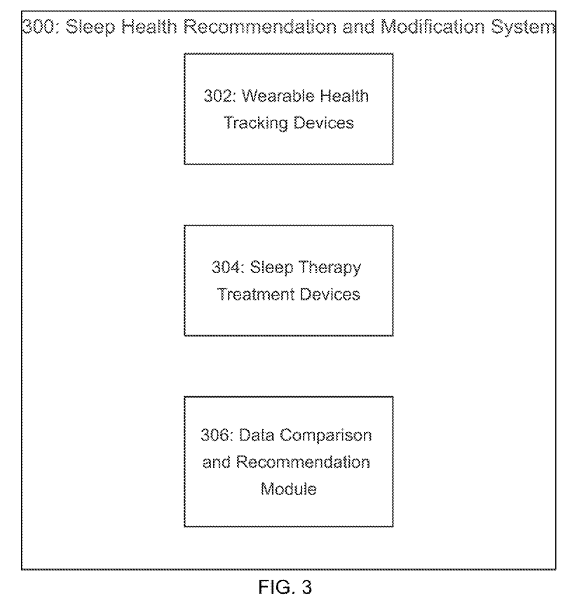

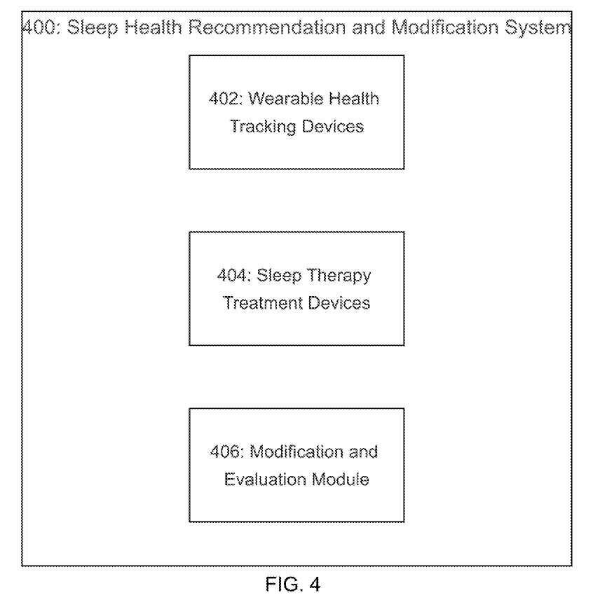

FIG. 3 is a diagram illustrating a system 300 including wearable health tracking devices. The wearable health tracking devices component may be utilized to obtain user health data. This component may include devices such as smart watches, smart rings, smart bands, earables, and noseables which may be capable of collecting various health metrics from the user. The data obtained from these devices may be combined with data from sleep therapy treatment devices, such as CPAP devices, to provide comprehensive insights into the user's health and sleep patterns.

Example wearable health tracking devices and their sensors include:

### Samsung Galaxy Watch

1. Heart Rate Monitor - Measures heart rate continuously.

2. Electrocardiogram (ECG) - Monitors heart rhythm.

3. Bioelectrical Impedance - Measures body composition, including body fat and muscle mass.

4. Accelerometer - Tracks movement and activity levels.

5. Gyroscope - Detects orientation and rotation.

6. Barometer - Measures atmospheric pressure for altitude changes.

7. GPS - Provides location tracking for outdoor activities.

8. Ambient Light Sensor - Adjusts display brightness based on surrounding light.

### Apple Watch

1. Heart Rate Sensor - Continuously monitors heart rate.

2. Electrocardiogram (ECG) - Allows users to take ECGs.

3. Blood Oxygen Sensor - Measures blood oxygen saturation (SpO2).

4. Accelerometer - Tracks movement and activity.

5. Gyroscope - Detects orientation and rotation.

6. Barometric Altimeter - Measures elevation changes.

7. GPS - Provides location tracking for workouts.

8. Ambient Light Sensor - Adjusts display brightness.

### Samsung Galaxy Ring

1. Heart Rate Monitor - Tracks heart rate continuously.

2. Sleep Tracking Sensors - Monitors sleep patterns and quality.

3. Body Temperature Sensor - Measures skin temperature.

4. SpO2 Sensor - Monitors blood oxygen levels.

5. Accelerometer - Detects movement and activity levels.

6. Gyroscope - Measures orientation and motion.

7. Bioelectrical Impedance Analysis (BIA) - Assesses body composition (if included).

### Oura Ring

1. Heart Rate Monitor - Continuously tracks heart rate.

2. Pulse Oximeter - Measures blood oxygen saturation (SpO2).

3. Temperature Sensor - Monitors body temperature variations.

4. Accelerometer - Tracks movement and activity.

5. Gyroscope - Detects orientation and motion.

6. Sleep Tracking Sensors - Monitors sleep stages and quality.

7. Activity Level Sensors - Measures daily activity and recovery.

Whoop Band which measures:

1. Heart rate: Live heart rate, resting heart rate, and current maximum heart rate

2. Respiratory rate: Respiratory rate during exercise and daily activity

3. Blood oxygen: Blood oxygen level, also known as SpO2

4. Skin temperature: Skin temperature

5. Sleep: Sleep duration and quality

6. Calories: Calories expended

7. Heart rate variability: Heart rate variability (HRV)

Earables are ear-worn wearable health tracking devices that may be used for health tracking, for example, for continuous monitoring of cardiovascular function; oxygen consumption and blood flow; and tracking eating episodes as well as dietary and swallowing activities. In some examples, earables may be confined with headphones. Ear-worn devices may be used to actively monitor a patient for sounds associated with airway obstructions and/or other pathologies. In one example, the ear-worn devices may include a plurality of microphone arrays to detect sounds. The ear-worn devices may analyze the sounds and determine a location of the sounds in the patient, e.g., using a position determination algorithm. The ear-worn devices may further determine that the sounds are associated with a pathology, such as an airway obstruction. In some embodiments, the ear-worn devices determine other attributes of the pathology, such as a type of the pathology (e.g., obstructive sleep apnea (OSA), a degree of the pathology (e.g., partial collapse, complete collapse, etc.).

In some embodiments, the ear-worn devices emit sounds or vibrations to detect pathologies. For example, the ear-worn devices may emit sounds and/or vibrations and identify reflections of the same from the ear canal, airway, or other part of the patient. The ear-worn devices may analyze the reflected sounds and/or vibrations to identify a pathology.

In some embodiments, the ear-worn devices use the sounds (whether emitted or detected) and/or vibrations to generate a model of the geometry of the ear canal of the wearer of the earbuds. The ear-worn devices may use the models to detect pathologies, e.g., based on stored models of the ear canal under various conditions (e.g., with an airway obstruction, etc.).

Another form of wearable health tracking device is a noseable which may be nose-worn by a patient.

In some forms of the present technology, a wearable health tracking device may have one or more sensors and/or actuators provided therein, for measurement of the patient’s physiological and sleep data. One or more sensors and one or more actuators may respectively be embedded within the wearable device, for example between textile layers of headgear or a wristband or chest band of the wearable device, or may be attached to internal and/or external surfaces of the wearable device.

In some forms, the wearable device may comprise one or more leads, cables, or other electrically conductive elements extending therefrom and being in electrical communication with one or more of the sensors or actuators. Each such electrically conductive element may comprise a terminal that can contact skin of the wearer of the device to provide one or more suitable signal grounding points on the face or head of the wearer, such as behind the ear, or below the eye socket. This may be useful for implementation of an EEG, EMG, or EOG system within the wearable device.

Sensors embedded in the device can help collect sleep-related data and physiological indicators such as vital data; this can be used, for example, for diagnostic purposes, and/or to determine an improvement in sleep and health by comparing data before and after start of a particular therapy, such as CPAP therapy, use of a mandibular advancement device, and/or cognitive behavioural therapy. This data can be processed and the patient informed of how the therapy is improving sleep. The data may be communicated to external computing devices such as a smartphone of the user, and/or a monitoring server that is operated by or accessible to a clinician or other healthcare provider.

With data-based feedback being obtained on how the therapy is actually helping (for example, via an application executing on the patient’s smartphone), patient compliance is more likely. The data collected may also be useful in determining population-level sleep and/or physiological characteristics of one or more cohorts of patients undergoing therapy, thereby potentially enabling better customisation of therapy to patients falling within particular categories.

In some forms, sensors and associated electronics may be integrated at least partly between fabric layers of a band of the wearable device. For example, various sensor/actuator modules and/or associated circuitry may lie between an inner, user-contacting, fabric layer, and an outer, non-user-contacting, fabric layer.

In some forms of the present technology, one or more sensor modules and/or actuator modules may be enclosed entirely between the fabric layers, such that no part of the one or more sensor modules is exposed. One example of a sensor module that may be fully embedded in this way is an accelerometer or gyroscope.

In some forms of the present technology, a sensor module or actuator module may be at least partly exposed. For example, a humidity sensor may be at least partly exposed to ambient through an outer fabric layer to measure humidity of the patient’s environment. To this end, the outer fabric layer may comprise an aperture through which a surface of the humidity sensor may be exposed. In another example, a sensor may have a surface thereof exposed through an inner fabric layer (e.g., through an aperture formed therein), such that the sensor surface can contact the skin of the user when the wearable device is worn by the user. The sensor may be a pulse oximeter, for example.

In some forms of the present technology, one or more electronic components (such as sensors or actuators) may be woven or otherwise integrated into material of a band of the wearable device, for example into an outer fabric layer or an inner fabric layer. This may enable distribution of a sensor over a larger area for more informative and/or accurate measurements to be made.

Some forms of the present technology may comprise one or more sensors, such as an accelerometer and/or gyroscope and/or magnetometer, for determining sleeping position and movements of a user. In some forms, the determined sleeping position and movements may be used to regulate the operation of a therapy device (such as a CPAP machine), and/or to provide a sensory stimulus to the user to cause them to change position. For example, if a number of apnea and/or hypopnea events above a certain threshold, and/or a decrease in blood oxygenation, is detected by the one or more sensors, this may be indicative of back sleeping. One or more actuators may receive an activation signal based on the detection, and the activation signal may cause the one or more actuators to generate a vibration or other tactile stimulus to irritate the user sufficiently to cause them to switch to another sleeping position.

Measurements recorded by an accelerometer may be used to determine the user’s sleeping position and to adjust therapy accordingly. For example, for a user on CPAP therapy, when it is detected that the user is lying on their back, the therapy pressure can be ramped up slowly by the CPAP machine to prevent sleep apnea events. When side sleeping is detected, the therapy pressure can be lowered. When an upright position is detected, the flow and pressure could be just sufficient to avoid a feeling of suffocation. In like fashion, measurements recorded by the gyroscope may be used to determine movements of the user and to adjust therapy accordingly. When a lot of movement is detected, indicating that the patient is likely awake, the therapy pressure can be kept low enough to avoid a feeling of suffocation. When the movements subside, the therapy pressure can be very slowly ramped up to avoid discomfort.

In some forms of the present technology, accelerometer and/or gyroscope measurements recorded by a wearable device may be used to determine a sleep stage of the user. Sleep stage data can then be used to provide feedback to the user, and/or to control the operation of an external device such as a CPAP machine. For example, it may be difficult for a CPAP user to fall asleep if therapy commences while they are still awake. Accordingly, the CPAP machine may remain in an “off” or paused state if the accelerometer and/or gyroscope measurements are indicative of an awake or light sleep stage, and then switched on (typically, with a gentle ramp-up) once measurements indicate that the user is in a deep sleep stage. Conversely, if therapy has already commenced and it is detected that the user has switched from deep to light sleep, where therapy may rouse the user, the CPAP machine may be paused until the user is in deep sleep again, for example.

In some forms of the present technology, a pulse oximeter incorporated in the wearable device may be used to assess sleep health. Measurements recorded by the pulse oximeter may be used to determine blood oxygen saturation level and heart rate during the period that the device is worn. This data may be transmitted to an external computing device such as a smartphone, other mobile computing device, or laptop or desktop computing system of the user or a clinician. The data may be consolidated to provide feedback to the patient on their health levels, and recommendations for follow-up (for example, by a clinician).

In one example, an Apnea Hypopnea Index (AHI), which is a measure that clinicians use to classify the severity of sleep apnea, may be determined based on sensor measurements. Computation of AHI may use a combination of data from different sensors, e.g. blood oxygen level and heart rate (for example, measured by a PPG sensor), and chest movement (for example, measured by an accelerometer and/or gyroscope). The AHI value may be used to determine when an “apnea” occurs.

Accordingly, by tracking the AHI over time, a clinician will be able to tell if the patient has sleep apnea, and provide details of how severe it is. Further, by analysis of the AHI data together with other sensor data, the clinician may be able to not only correlate the frequency of apneas with particular sleeping positions (e.g. sleeping on back or sleeping on side), but also to adjust therapy (e.g. CPAP therapy) to the specific needs of the patient. For example, for a user on CPAP therapy, the amount of mouth breathing may be detected using temperature and/or humidity sensors located inside a plenum chamber of a patient interface, and a nasal or full-face mask prescribed accordingly (with full-face masks being more suitable for mouth breathers). Additionally, pressure generator settings that will produce a flow rate most suitable for the user may be recommended based on the sensor measurements. For example, a clinician may prescribe higher pressure settings (or equivalently, higher flow rates) for patients with a high detected rate of apnea or hypopnea events. The prescribed flow rate may also depend on the anatomical structure of the patient, for example if the patient has a more collapsible upper airway.

In some forms of the present technology, an EEG sensor may be provided in a wearable device. Typically, the EEG sensor comprises a plurality of EEG electrodes that generate signals that may be analysed to detect sleep stages. The signals may be transmitted to an external device, such as the user’s smartphone, and the sleep stage, cycle and duration information may be used to provide feedback to the patient on their sleep health and/or how well sleep therapy is progressing, as well as recommendations for enhanced health. For example, the EEG sensor measurements may be used for accurate sleep staging, to enable a more accurate determination of when an apnea or arousal from sleep occurs, e.g. during a sleep study.

In some forms of the present technology, the sleep stage information may be used to activate sleep-enhancing white/pink noise, and/or binaural beats. These may be produced by audio devices embedded in the wearable device itself, or by external devices that receive trigger signals from the wearable device. For example, if the wearable device comprises headgear, one or more miniature bone-conduction speakers may be incorporated at temple regions of the headgear.

In some forms of the present technology, a wearable device may incorporate electromyography (EMG) and/or electrooculography (EOG) sensors. EMG and EOG sensor signals may be analysed to determine REM sleep stage occurrences. In similar fashion to examples that incorporate EEG sensors, the sleep stage information determined by the EMG/EOG sensors may be used to provide feedback to the patient on their sleep health and/or how well sleep therapy is progressing, or to activate one or more audio devices to produce sleep-enhancing noise.

In some forms of the present technology, a microphone, such as a MEMS microphone or Electret microphone, may be incorporated in a wearable device to detect snoring. Alternatively, another device external to the wearable device may comprise such a microphone.

In some forms of the present technology, a combination of sensors and actuators may be provided to effect localised temperature change to improve patient comfort. For example, an EEG sensor and/or pulse oximeter (PPG) may be provided in the wearable device, together with a temperature sensor and/or a humidity sensor. Signals from the EEG and/or PPG sensors may be analysed to detect sleep state, and signals from the temperature sensor and/or humidity sensor may be used to assess ambient comfort levels. One or more Peltier elements may be provided, for example in wearable form on a wristband, and may be coupled to circuitry that communicates with the EEG/PPG and temperature/humidity sensors to receive signals indicative of sleep state and ambient comfort level, and that causes the Peltier element to be activated to locally warm or cool the body (e.g. at the wrist) to help the patient remain in a comfortable sleep state.

In some forms of the present technology, a haptic feedback element (such as a miniature vibratory motor) may be incorporated in the wearable device, for example in a temple region of a headgear of the wearable device. The haptic feedback element may deliver vibrations to the patient to produce a calming effect. For example, heart rate data from a pulse oximeter of the wearable device may be monitored, and if this exceeds a threshold, transmit a trigger signal to the haptic feedback element to cause it to vibrate at a few beats lower than the user’s current heart rate, to help slow it down. In another example, a haptic feedback element may be used to influence the sleeping position of the user if it is detected that they are in a sleeping position that is correlated with apnea or hypopnea events.

In some forms of the present technology, one or more miniature thermo electric generators (TEGs) may be incorporated into the wearable device, such that the difference between the patient’s body temperature and the ambient temperature may be used to generate a potential difference and thus to provide power to the various electronic components (sensors, actuators, processor, etc.) of the wearable device.

In some forms of the present technology, multiple sensors may be incorporated in a single module. For example, an accelerometer and/or gyroscope and/or magnetometer may be incorporated in a single package (as an inertial measurement unit, IMU, or inertial and magnetic measurement unit, IMMU).

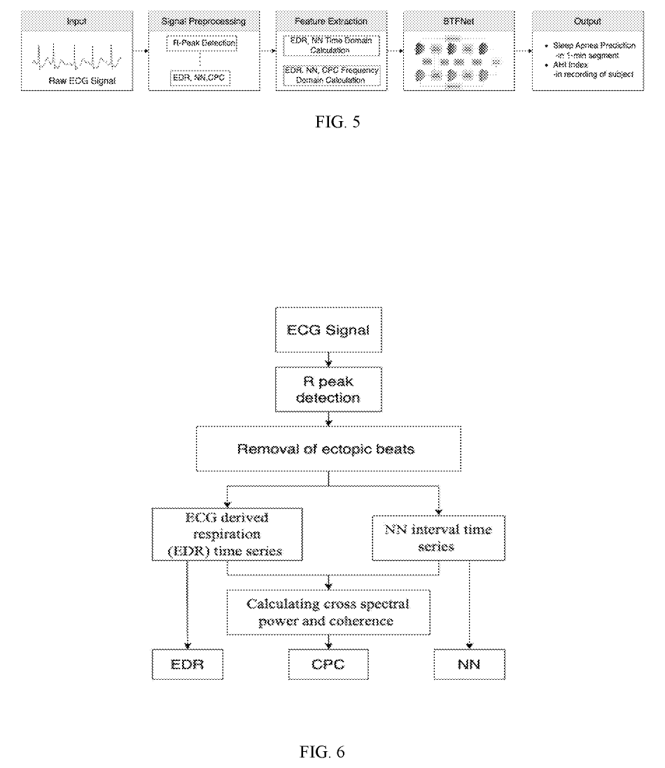

In some forms of the technology, and ECG-Based Sleep Apnea Detection Procedure is provided. A form of a pipeline for detecting sleep apnea using ECG signals is shown in FIG. 5, and follows a structured approach, as illustrated in the image. An overview of each step in the process is:

### 1. Input (Raw ECG Signal)

The pipeline begins with the acquisition of raw ECG data. This signal serves as the foundational input for subsequent processing and analysis.

### 2. Signal Preprocessing

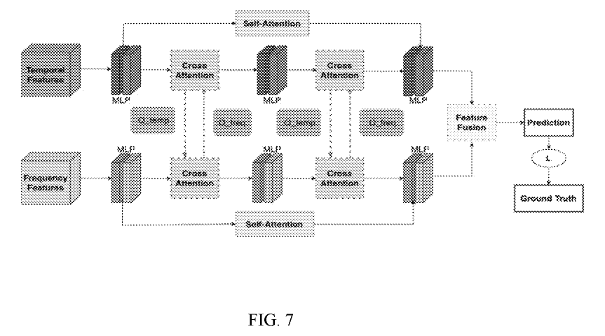

The raw ECG signal undergoes preprocessing steps as shown in FIG. 6 to enhance its quality and extract relevant physiological markers. Key components include:

R-Peak Detection: Identifying the R-peaks within the ECG signal, which is crucial for heart rate analysis and other derived features.

EDR, NN, and CPC Computation: Deriving ECG-Derived Respiration (EDR), normal-to-normal (NN) intervals, and Cardiopulmonary Coupling (CPC), which are indicative of respiratory and cardiovascular interactions.

### 3. Feature Extraction

Following preprocessing, various features are extracted from both the time and frequency domains to capture comprehensive information on sleep dynamics:

Time Domain Calculations: Extracting EDR and NN features in the time domain to identify patterns related to respiratory and heart rate variability.

Frequency Domain Calculations: Computing EDR, NN, and CPC in the frequency domain to uncover spectral characteristics that may signify sleep apnea events.

### 4. BTFNet (Biotemporal Frequency Network)

The extracted features are fed into the BTFNet model as shown in FIG. 7, a specialized deep learning architecture designed to integrate time-domain and frequency-domain features through self-attention and cross-attention mechanisms. This allows the model to learn complex dependencies across feature domains, improving the precision of sleep apnea detection.

### 5. Output

The final output consists of sleep apnea predictions and relevant indices:

Sleep Apnea Prediction: Classifying each 1-minute segment of the ECG signal as apnea or non-apnea.

In some forms of the technology, the above technique may be adapted to other signal modalities, for example, an accelerometer that contains pulse/heartrate information, pressure, flow, PPG signals that contain heart rate information, and/or pulse amplitude information, may be used to derive a respiratory signal and look for cardiopulmonary coupling.

The wearable health tracking devices component may play a role in the system by enabling the collection of user health data, which may then be used in conjunction with sleep therapy data to generate combined user data. This combined data may be instrumental in providing personalized recommendations for improving sleep health. The recommendations may include sleep condition screening, potential sleep therapy treatments, or sleep hygiene actions.

The data obtained from the wearable health tracking devices component may also be compared with health data from other users to make personalized recommendations. This comparison may help in identifying users with similar characteristics and tailoring recommendations accordingly. Additionally, the data may be used to suggest or make changes to sleep therapy treatment parameters, either while the user is using a sleep therapy treatment device or not.

Furthermore, the wearable health tracking devices component may contribute to evaluating the effectiveness of sleep therapy treatments or assessing the quality of the user's sleep. This evaluation may be crucial in determining the success of the therapy and making necessary adjustments to improve outcomes. The component may also be involved in monitoring the effectiveness of sleep therapy when the user is using a device without electronic monitoring capabilities, such as a mouthguard.

Overall, the wearable health tracking devices component may serve as an element in the system, enabling the collection and analysis of user health data to enhance sleep health through personalized recommendations and treatment modifications.

The sleep therapy treatment devices component may obtain user sleep therapy data, may enable generating personalized recommendations for improving sleep health. This component may include devices such as CPAP devices, which may be used to gather data on the user's sleep therapy treatment. The data obtained from these devices may be combined with data from wearable health tracking devices to provide comprehensive insights into the user's sleep patterns and therapy effectiveness. The combination of data may allow for the generation of personalized recommendations, which may include sleep condition screening, possible sleep therapy treatments, or sleep hygiene actions. Furthermore, the data from the sleep therapy treatment devices component may be used to generate modifications to sleep therapy treatment parameters, ensuring that the therapy is tailored to the user's specific needs. This process may involve evaluating the effectiveness of the current sleep therapy treatment and making necessary adjustments to optimize the user's sleep health. The integration of data from both wearable health tracking devices and sleep therapy treatment devices may provide a holistic view of the user's sleep health, enabling more accurate and effective recommendations and modifications.

The data comparison and recommendation module, identified as a component, may serve as an element in the system by facilitating the comparison of user health data with health data from other users. This module may utilize data obtained from wearable health tracking devices and sleep therapy treatment devices to provide personalized recommendations. The module may compare the user's data with data from other users to make recommendations for screening or possible treatments. This comparison may be instrumental in identifying users with similar characteristics, thereby enabling the system to tailor recommendations effectively. The module may also refer users to a marketplace of sleep therapy providers or device providers based on these recommendations. Additionally, the module may suggest a specific provider or rank providers based on the recommendations, thereby enhancing the user's access to suitable sleep therapy solutions. The actions associated with this module, such as comparing data and providing recommendations, may be seamlessly integrated with the system's overall functionality, ensuring that users receive personalized and effective sleep health recommendations. The module's ability to correlate data from various sources may enhance the accuracy and relevance of the recommendations provided, thereby contributing to improved sleep health outcomes for users.

The wearable health tracking devices component may serve as an element in obtaining user health data. This component may be designed to interface with various wearable health tracking devices, which may include smart watches, smart rings, or smart bands. The primary function of this component may involve the acquisition of user health data, which may then be utilized in conjunction with data from sleep therapy treatment devices. The data obtained may be instrumental in generating modifications to sleep therapy treatment parameters and evaluating the effectiveness of sleep therapy treatments.

The wearable health tracking devices component may work in tandem with the modification and evaluation module to facilitate the generation of modifications to treatment parameters. This process may involve the combination of user health data with sleep therapy data to produce combined user data. The combined data may then be analyzed to generate modifications that may be applied to sleep therapy devices, such as CPAP devices. Additionally, the component may contribute to the evaluation of the effectiveness of sleep therapy treatments by assessing the quality of the user's sleep.

The component may also play a role in suggesting or making changes to sleep therapy treatment parameters based on the user health data obtained while the user is using a sleep therapy treatment device. This capability may allow for real-time adjustments to be made to the treatment parameters, potentially enhancing the effectiveness of the therapy. Furthermore, the component may be involved in monitoring the effectiveness of sleep therapy when the user is using a device without electronic monitoring capabilities, such as a mouthguard. In such cases, the wearable health tracking devices may provide valuable data that may be used to assess the therapy's effectiveness and/or may recommend additional treatments.

Overall, the wearable health tracking devices component may be integral to the system's ability to provide personalized recommendations for improving sleep health. By obtaining and analyzing user health data, this component may enable the system to generate modifications to treatment parameters, evaluate treatment effectiveness, and suggest changes to therapy treatments, thereby contributing to the overall goal of enhancing sleep health outcomes.

The sleep therapy treatment devices component may be responsible for obtaining user sleep therapy data. This component may include devices such as CPAP devices, which may be utilized to gather data pertinent to the user's sleep therapy. The data obtained from these devices may be combined with data from wearable health tracking devices to generate modifications to sleep therapy treatment parameters. The combination of data may allow for the generation of personalized recommendations for improving sleep health. These recommendations may include sleep condition screening, possible sleep therapy treatments, or sleep hygiene actions. The component may also facilitate the evaluation of the effectiveness of sleep therapy treatments or the quality of the user's sleep. The data obtained may be used to suggest or make changes to sleep therapy treatment parameters, potentially enhancing the user's sleep health. Additionally, the component may monitor the effectiveness of sleep therapy when the user is using a device without electronic monitoring capabilities, such as a mouthguard, by utilizing data from wearable health tracking devices. The sleep therapy treatment devices component may thus play a role in the overall system by providing data that may be used to tailor sleep therapy treatments to the user's specific needs.

The modification and evaluation module, identified as a component, may serve as an element in the system by facilitating the generation of modifications to treatment parameters and evaluating the effectiveness of sleep therapy treatments. This module may interact with wearable health tracking devices and sleep therapy treatment devices to achieve its objectives. The module may generate modifications to sleep therapy treatment parameters by analyzing data from wearable health tracking devices and sleep therapy treatment devices. This process may involve combining data to provide recommendations for sleep condition screening, possible sleep therapy treatments, or sleep hygiene actions. The module may also evaluate the effectiveness of a sleep therapy treatment or the quality of a user's sleep by assessing data obtained from wearable health tracking devices. This evaluation may be crucial for determining the success of the therapy and making necessary adjustments. Additionally, the module may suggest or make changes to sleep therapy treatment parameters based on the user health data obtained from wearable health tracking devices while the user is using a sleep therapy treatment device. This capability may ensure that the treatment is tailored to the user's current health status and needs. Furthermore, the module may monitor the effectiveness of sleep therapy when the user is using a sleep therapy device without electronic monitoring capabilities, such as a mouthguard, by utilizing data from wearable health tracking devices. This monitoring may provide insights into the therapy's impact and guide further modifications. The modification and evaluation module, therefore, may play a role in enhancing the personalization and effectiveness of sleep therapy treatments by leveraging data from various devices and making informed modifications.

In further examples, additional health data may be obtained from non-wearable health devices such as digital scales; exercise machines such as treadmills, stationary bikes, step machines, stair climbers, elliptical machines, or rowing machines; bicycle smart trainers; and bicycle head units such those produced by Garmin or Wahoo, or from software applications that aggregate health data from other sources.

In further examples, data from other sensors may also be used. For example, WO 2022024046 A1, the disclosure of which is incorporated herein by reference, describes how additional sensors may be used to supplement data from a sleep treatment device. These sensors can include environmental sensors that monitor room temperature, humidity, and noise levels, which can impact sleep quality. Additionally, sensors that track light exposure and air quality may also be utilized to provide a more comprehensive understanding of the factors affecting sleep. By integrating data from these additional sensors, the system can offer more precise recommendations and modifications to sleep therapy treatments, further enhancing the user's sleep health outcomes.

In some examples, the wearable health tracking devices may be used for ongoing monitoring of the patient. For example, to determine how are therapies effecting sleep, e.g. weight loss drugs, lifestyle interventions, potentially slow response as a sleep therapy. If the response is slow might suggest complimentary therapies are appropriate, could be used to indicate when complimentary therapies can be ceased.

In some examples, the use of wearable health tracking devices may be used to obtain data at multiple sensing locations. In some example, this may enable additional measures to be obtained, for example, to extract pulse wave timing (time the pulse passes the ear v wrist v finger using an earbud, watch, ring etc or other devices at other locations) which may be related to arterial wall stiffness, related to stress, or stress response to OSA, insomnia, alcohol consumption, other medical conditions.

In other example, peripheral and core temperature estimates, or the difference between, may be valuable for circadian timing estimates, sleep onset detection (corresponding with the maximum difference between core and peripheral temperature, as heat is lost from the core leading to sleep onset).

In other examples, wearable health tracking devices may be used to monitor both the user and their environment, closing the loop on environmental conditions impact on sleep, light, temperature, social media, noise, partner’s sleep, etc.

In other examples, suitable wearable health tracking devices, such as smart watches may be used to capture feedback on the quality of a user's sleep by presenting a question to the user on waking. In some example, the may be used to provide feedback such as coaching, or facilitating primary care conversations. For example, a wearable device may output a message suggesting the patient should discuss sleep.

In some examples, a diagnostic tool, such as a generative artificial intelligence software tool may use the health data. In an example, the software tool acts as a sleep clinician but is guided by shared historic wearable data. When the software tool is started, it asks for access to wearable/health data, performs analysis on the data, then will ask further questions about sleep/health/lifestyle wherein the questions are derived from wearable data. Things like if there's sleep irregularity noticed, "Are you a shift worker?" or even more open ended questions "Do you struggle with your sleep?", or asking more direct questions that would shed a lot of light "Do you take any medication regularly?" or "Have you been diagnosed with a sleep disorder or suspect you might have one? If so, what?". The software tool may then output pathways to consider, and follows up by monitoring wearable data when a person follows the advice or uses the therapy.

In some examples, data from the wearable health tracking device may be used to enable a sleep specialist to decide on a diagnosis technique, for example, to identify cases that are most likely to be better suited to in sleep laboratory testing (PSG) or home sleep testing (HST), based on complexity of sleep issues, e.g. does the sleep (and wakeful) data look like someone who could easily be diagnosed and successfully treated going from HST to therapy, or do things look more complicated indicating PSG and lab observation might be more appropriate?

In some examples, data from the wearable health tracking device may be used to categorise patients into risk categories. In some examples, patients determined to be at low risk may be directed to one or more low risk sleep interventions such as pillows, a sleep coach (human, or AI) or dietician and the wearable heath tracking device used to monitor the effectiveness of the intervention and/or to reevaluate the risk category.

The system described above may be extended to operate more generally with any respiratory therapy system suitable for delivering any type of respiratory therapy including, but not limited to, continuous positive airway pressure (CPAP) therapy, non-invasive ventilation (NIV), invasive ventilation (IV), high flow therapy (HFT), oxygen concentration and ventilation. A range of respiratory disorders exist. Certain disorders may be characterised by particular events, e.g. apneas, hypopneas, and hyperpneas. Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders. Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders. Respiratory pressure therapy is the application of a supply of air to an entrance to the airways at a controlled target pressure that is nominally positive with respect to atmosphere throughout the patient’s breathing cycle (in contrast to negative pressure therapies such as the tank ventilator or cuirass). CPAP, NIV and IV are examples of respiratory pressure therapy. Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient’s airways is ‘open’ (unsealed) and the respiratory therapy may only supplement the patient’s own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient’s peak inspiratory flow rate. HFT has been used to treat OSA, CSR, respiratory failure, COPD, and other respiratory disorders. As an alternative to constant flow rate, the treatment flow rate may follow a profile that varies over the respiratory cycle.

Another form of flow therapy is long-term oxygen therapy (LTOT) or supplemental oxygen therapy. For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

Another form of respiratory therapy is oxygen concentration. An oxygen concentrator is a device that concentrates the amount of oxygen in a gas supply to provide an oxygen-enriched flow of breathable gas to a patient. Some forms of oxygen concentrators operate by taking ambient air and selectively reducing its nitrogen content to produce the oxygen-enriched flow of breathable gas.

Another form of respiratory therapy is ventilation. A ventilator is a device that causes breathable air to move into and/or out of the lungs to enable a patient to breathe where the patient is unable to breathe themselves, or requires assistance to do so. A ventilator creates the flow of air through a mechanical mechanism.

These respiratory therapies may be provided by a respiratory therapy system or device. Such systems and devices may also be used to screen, diagnose, or monitor a condition without treating it. A respiratory therapy system may comprise a Respiratory Pressure Therapy Device (RPT device), an air circuit, a humidifier, a patient interface, an oxygen source, and data management.

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g.,with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about10 cmH2O. For flow therapies such as nasal HFT, the patient interface is configured to insufflate the nares but specifically to avoid a complete seal. One example of such a patient interface is a nasal cannula.

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. The flow of air may be pressurised. Examples of RPT devices include a CPAP device, NIV device, HFT device, oxygen concentrator, and a ventilator.

In some examples, a sleep health recommendation and modification system may generally include one or more of servers, one or more communication networks, and one or more computing devices. The server and computing device may also be in communication with one or more respiratory therapy devices via the one or more communication networks. The server and computing device may also be in communication with one or more wearable health tracking devices via the oneor more communication networks. In some examples, this communication with wearable health tracking devices may be indirect, for example, via a mobile device or a computing device to which the wearable health tracking device is connected. The one or more communication networks may comprise, for example, the Internet, a local area network, a wide area network and/or a personal area network implemented over wired communication network(s), wireless communication network(s), or a combination thereof (for example, a wired network with a wireless link). In one form, local communication networks may utilize one or more communication standards, such as Bluetooth, Near-Field Communication (NFC), or a consumer infrared protocol.

An embodiment provides a system for generating modifications to sleep therapy treatment parameters using data from wearable health tracking devices and sleep therapy treatment devices, the system comprising:

one or more processors; and a memory storing instructions that, when executed by the one or more processors, cause the system to:

obtain user health data from one or more wearable health tracking devices;

obtain user sleep therapy data from one or more sleep therapy treatment devices;

combine the user health data and the user sleep therapy data to generate combined user data;

generate modifications to sleep therapy treatment parameters based on the combined user data; and apply the modifications to a sleep therapy device.

In an embodiment, the instructions further cause the system to evaluate effectiveness of sleep therapy treatment or quality of user's sleep using the user health data obtained from the one or more wearable health tracking devices.

In an embodiment, the one or more wearable health tracking devices include at least one of a smart watch, a smart ring, or a smart band.

In an embodiment, wherein the one or more sleep therapy treatment devices include a continuous positive airway pressure (CPAP) device.

In an embodiment, the instructions further cause the system to suggest or make changes to sleep therapy treatment parameters based on the user health data obtained from the one or more wearable health tracking devices while the user is using a sleep therapy treatment device.

In an embodiment, the instructions further cause the system to suggest or make changes to sleep therapy treatments based on the user health data obtained from the one or more wearable health tracking devices while the user is not using a sleep therapy treatment device.

In an embodiment ,the instructions further cause the system to suggest an additional sleep therapy treatment based on the user health data obtained from the one or more wearable health tracking devices while the user is using a sleep therapy treatment device.

In an embodiment, the instructions further cause the system to monitor effectiveness of sleep therapy when the user is using a sleep therapy device without electronic monitoring capabilities by using the user health data obtained from the one or more wearable health tracking devices.

The server may comprise processing facilities represented by one or more processors, memory, and other components typically present in such computing environments. The processing capabilities of the processor may be provided, for example, by one or more general-purpose processors, one or more special-purpose processors, or cloud computing services providing access to a shared pool of computing resources configured in accordance with desired characteristics, service models, and deployment models. In the example illustrated the memory stores information accessible by processor, the information including instructions that may be executed by the processor and data that may be retrieved, manipulated or stored by the processor. The memory may be of any suitable means known in the art, capable of storing information in a manner accessible by the processor, including a computer readable medium, or other medium that stores data that may be read with the aid of an electronic device.

Although the processor and memory are illustrated as being within a single unit, it should be appreciated that this is not intended to be limiting, and that the functionality of each as herein described may be performed by multiple processors and memories, that may or may not be remote from each other and the remainder of system.

The instructions may include any set of instructions suitable for execution by the processor. For example, the instructions may be stored as computer code on the computer readable medium. The instructions may be stored in any suitable computer language or format. Data may be retrieved, stored or modified by processor in accordance with the instructions. The data may also be formatted in any suitable computer readable format. Again, while the data is illustrated as being contained at a single location, it should be appreciated that this is not intended to be limiting – the data may be stored in multiple memories or locations. The data may include one or more databases.

In some examples, the server may communicate one-way with computing device(s) by providing information to one or more of the computing devices, or vice versa. In other embodiments, server and computing device(s) may communicate with each other two-way and may share information and/or processing tasks.

The computing device(s) can be any suitable processing device such as, without limitation, a personal computer such as a desktop or laptop computer, or a mobile computing device such as a smartphone or tablet. Computing device may include one or more processors. Computing device may also include memory/data storage, input/output (I/O) device, and communication interface.

The one or more processors can include functional components used in the execution of instructions, such as functional components to fetch control instructions from locations such as memory/data storage, decode program instructions, and execute program instructions, and write results of the executed instructions. Memory/data storage may be the computing device's internal memory, such as RAM, flash memory or ROM. In some examples, memory/data storage may also be external memory linked to computing device, such as an SD card, USB flash drive, optical disc, or a remotely located memory (e.g. accessed via a server such as server), for example. In other examples, memory/data storage can be a combination of external and internal memory.

Memory/data storage includes processor control instructions and stored data that instruct processor to perform certain tasks, as described herein. As noted above, in examples instructions may be executed by, and data stored in and/or accessed from, resources associated with the server in communication with the computing device.

In examples, the input/output (I/O) devices may include one or more displays. In examples, the display may be a touch sensitive screen allowing for user input in addition to outputting visible information to a user of computing device. In examples, I/O devices may include other output devices, including one or more speakers, and haptic feedback devices.

In examples, the input/output (I/O) devices may include input devices such as physical input devices (for example, buttons or switches), optical sensors (for example, one or more imaging devices such as a camera), and inertial sensors (particularly in examples where the computing device is a mobile computing device). It will be appreciated that other I/O devices may be included, or otherwise accessed through an I/O interface (for example, interfacing with peripheral devices connected to the computing device ). A communication interface enables computing device to communicate via the one or more networks.

This specification includes flow diagrams indicating methods implementable, at least in part, by system in certain forms of the technology. The flow diagrams are representative of example computer readable instructions for implementing the exemplary methods described herein. In examples, the computer readable instructions comprise one or more algorithms for execution by one or more of the processors, for example processors, described herein. The instructions for performing these functions are, optionally, included in a non-transitory computer readable storage medium, for example memory , or other computer program product configured for execution by one or more processors. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A computer readable storage medium, as used herein, is not to be construed as being transitory signals per se, such as radio waves or other freely propagating electromagnetic waves, electromagnetic waves propagating through a waveguide or other transmission media, or electrical signals transmitted through a wire. However, persons of ordinary skill in the art will readily appreciate that the entire algorithm and/or parts thereof can alternatively be executed by a device other than a processor and/or embodied in firmware or dedicated hardware in a well-known manner, e.g., it may be implemented by an application specific integrated circuit (ASIC), a programmable logic device (PLD), a field programmable logic device (FPLD), a field programmable gate array (FPGA), discrete logic, etc. For example, any or all of the components can be implemented by software, hardware, and/or firmware. Further, although the example algorithms are described with reference to the illustrated flowcharts, persons of ordinary skill in the art will readily appreciate that many other methods of implementing the example processor readable instructions may alternatively be used. For example, the order of execution of the blocks may be changed, and/or some of the blocks described may be changed, eliminated, or combined. As used herein the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor, a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a processor readable medium; or a combination thereof.

What is claimed is:

1. A method for providing personalized recommendations for improving sleep health using data from wearable health tracking devices and sleep therapy treatment devices, the method comprising:
obtaining, by a system, user health data from one or more wearable health tracking devices;
obtaining, by the system, user sleep therapy data from one or more sleep therapy treatment devices;
combining, by the system, the user health data and the user sleep therapy data to generate combined user data; and
providing, by the system, personalized recommendations for improving sleep health based on the combined user data, wherein the personalized recommendations include at least one of sleep condition screening, at least one sleep therapy treatment, at least one additional sleep therapy treatment, or at least one sleep hygiene action.

2. A method for generating modifications to sleep therapy treatment parameters using data from wearable health tracking devices and sleep therapy treatment devices, the method comprising:
obtaining, by a system, user health data from one or more wearable health tracking devices;
obtaining, by the system, user sleep therapy data from one or more sleep therapy treatment devices;
combining, by the system, the user health data and the user sleep therapy data to generate combined user data;
generating, by the system, modifications to sleep therapy treatment parameters based on the combined user data; and
applying, by the system, the modifications to a sleep therapy device.

3. The method of claim 2, further comprising evaluating, by the system, effectiveness of sleep therapy treatment or quality of user's sleep using the user health data obtained from the one or more wearable health tracking devices.

4. The method of claim 1, wherein the one or more wearable health tracking devices include at least one of a smart watch, a smart ring, or a smart band.

5. The method of claim 1, wherein the one or more sleep therapy treatment devices include a continuous positive airway pressure (CPAP) device.

6. The method of claim 1, further comprising comparing the user health data with health data obtained from other users of sleep therapy treatment devices to make personalized recommendations.

7. The method of claim 6, further comprising referring the user to a marketplace of sleep therapy providers or sleep therapy device providers based on the personalized recommendations.

8. The method of claim 7, further comprising suggesting a specific provider based on the personalized recommendations.

9. The method of claim 7, further comprising ranking providers based on the personalized recommendations.

10. The method of claim 2, wherein the one or more wearable health tracking devices include at least one of a smart watch, a smart ring, or a smart band.

11. The method of claim 2, wherein the one or more sleep therapy treatment devices include a continuous positive airway pressure (CPAP) device.

12. The method of claim 2, further comprising suggesting or making changes to sleep therapy treatment parameters based on the user health data obtained from the one or more wearable health tracking devices while the user is using a sleep therapy treatment device.

13. The method of claim 2, further comprising suggesting or making changes to sleep therapy treatments based on the user health data obtained from the one or more wearable health tracking devices while the user is not using a sleep therapy treatment device.

14. The method claim 2, further comprising suggesting an additional sleep therapy treatment based on the user health data obtained from the one or more wearable health tracking devices while the user is using a sleep therapy treatment device.

15. The method of claim 2, further comprising monitoring effectiveness of sleep therapy when the user is using a sleep therapy device without electronic monitoring capabilities by using the user health data obtained from the one or more wearable health tracking devices.

16. A system for providing personalized recommendations for improving sleep health using data from wearable health tracking devices and sleep therapy treatment devices, the system comprising:
one or more processors; and a memory storing instructions that, when executed by the one or more processors, cause the system to:
obtain user health data from one or more wearable health tracking devices;
obtain user sleep therapy data from one or more sleep therapy treatment devices;
combine the user health data and the user sleep therapy data to generate combined user data; and
provide personalized recommendations for improving sleep health based on the combined user data, wherein the personalized recommendations include at least one of sleep condition screening, at least one sleep therapy treatment, at least one additional sleep therapy treatment, or at least one sleep hygiene action.

17. The system of claim 16, wherein the one or more wearable health tracking devices include at least one of a smart watch, a smart ring, or a smart band.

18. The system of claim 16, wherein the one or more sleep therapy treatment devices include a continuous positive airway pressure (CPAP) device.

19. The system of claim 16, wherein the instructions further cause the system to compare the user health data with health data obtained from other users of sleep therapy treatment devices to make personalized recommendations.

20. The system of claim 19, wherein the instructions further cause the system to refer the user to a marketplace of sleep therapy providers or sleep therapy device providers based on the personalized recommendations.
